(19) United States
(12) Patent Application Publication
(10) Pub. No.: US 2014/0147863 A1
(43) Pub. Date: May 29, 2014

(54) METHODS AND DEVICES FOR DIAGNOSING ALZHEIMERS DISEASE

(71) Applicants: Sidney E O'Bryant, Aledo, TX (US); Robert Clinton Barber, Benbrook, TX (US); Ramon Diaz-Arrastia, Olney, MD (US); Guanghua Xiao, Austin, TX (US); Peirrie Milton Adams, Dallas, TX (US); Joan Snavely Reisch, Dallas, TX (US); Rachelle Smith Doody, Austin, TX (US); Thomas John Fairchild, Fort worth, TX (US); Ralph L. McDade, Austin, TX (US); Samuel T. Labrie, Austin, TX (US)

(72) Inventors: Sidney E O'Bryant, Aledo, TX (US); Robert Clinton Barber, Benbrook, TX (US); Ramon Diaz-Arrastia, Olney, MD (US); Guanghua Xiao, Austin, TX (US); Peirrie Milton Adams, Dallas, TX (US); Joan Snavely Reisch, Dallas, TX (US); Rachelle Smith Doody, Austin, TX (US); Thomas John Fairchild, Fort worth, TX (US); Ralph L. McDade, Austin, TX (US); Samuel T. Labrie, Austin, TX (US)

(73) Assignee: RULES-BASED MEDICINE, INC., Austin, TX (US)

(21) Appl. No.: 13/697,978

(22) PCT Filed: May 13, 2011
(86) PCT No.: PCT/US11/36496

§ 371 Date: Jun. 19, 2013

Related U.S. Application Data
(60) Provisional application No. 61/334,955, filed on May 14, 2010.

(51) Int. Cl. G01N 33/543

(52) U.S. Cl. CPC G01N 33/54306
USPC 435/7.92

(57) ABSTRACT

Methods and devices for predicting, diagnosing, monitoring, or determining Alzheimer's disease in a human are described. In particular, methods and devices for predicting diagnosing, monitoring, or determining AD using measured concentrations of a combination of three or more analytes in a test sample taken from the human are described.

### FIELD OF THE INVENTION

The invention encompasses methods and devices for predicting, diagnosing, monitoring, or determining alzheimer's disease (AD) in a human.

### BACKGROUND OF THE INVENTION

Alzheimer's disease (AD) is the most common form of age-related dementia and one of the most serious health problems in the industrialized world. Current state-of-the art diagnostics rely on a synthesis of information obtained from a multidisciplinary team, typically consisting of a medical examination by specialists (neurologist, psychiatrist, or geriatrician), neuropsychological evaluation, clinical blood work, and neuroimaging. Even though this diagnostic scheme has been demonstrated as valid, it is time consuming, expensive, and relies on several specialists, whom are not always available.

An alternative approach would be to use biomarkers. Attempts to identify a single biomarker indicative of AD have been unsuccessful, although panels of biomarkers that achieve a correct classification rate of AD of over 90% have been described. However, these panels of biomarkers are derived from cerebrospinal fluid (CSF). CSF-based tests are generally invasive and not universally available. Ideally, a biomarker or a panel of biomarkers would be gleaned from blood, either serum or plasma. To date, however, there is no blood-based biomarker or panel of biomarkers that yields adequate diagnostic accuracy in AD.

Therefore, there is a need in the art for a fast, simple, reliable, non-invasive and sensitive method of predicting, diagnosing, monitoring, or determining AD. In a clinical setting, the early detection of AD would help medical practitioners to diagnose and treat AD more quickly and effectively. In addition, the detection of the early signs of AD would be useful as a measure of therapeutic efficacy of potential drugs that can treat AD.

### SUMMARY OF THE INVENTION

The present invention provides methods and devices for predicting, diagnosing, monitoring, or determining Alzheimer's disease in a human. In particular, the present invention provides methods and devices for predicting, diagnosing, monitoring, or determining Alzheimer's disease using measured concentrations of a combination of three or more analytes in a test sample taken from the human.

One aspect of the present invention provides a method of predicting, diagnosing, monitoring, or determining Alzheimer's disease in a human, the method comprising, providing a test sample comprising a sample of bodily fluid taken from the human, determining the concentrations of three or more sample analytes in a panel of biomarkers in said sample, wherein the sample analytes are selected from the group consisting of the biomarkers in Table A, and calculating a risk score for the human using the concentrations of three or more sample analytes in the panel of biomarkers in said sample, wherein the risk score represents the probability that the human has Alzheimer's disease.

In another aspect, the present invention provides a method of predicting, diagnosing, monitoring, or determining Alzheimer's disease in a human, the method comprising, providing a test sample comprising a sample of bodily fluid taken from the human, determining the concentrations of three or more sample analytes in a panel of biomarkers in said sample, wherein the sample analytes are selected from the group consisting of the biomarkers in Table B, and calculating a risk score for the human using the concentrations of three or more sample analytes in the panel of biomarkers in said sample, wherein the risk score represents the probability that the human has Alzheimer's disease.

In yet another aspect, the present invention provides a method of predicting, diagnosing, monitoring, or determining Alzheimer's disease in a human, the method comprising, providing a test sample comprising a sample of bodily fluid taken from the human, determining the concentrations of three or more sample analytes in a panel of biomarkers in said sample, wherein the sample analytes are selected from the group consisting of the biomarkers in Table C, and calculating a risk score for the human using the concentrations of three or more sample analytes in the panel of biomarkers in said sample, wherein the risk score represents the probability that the human has Alzheimer's disease.

In still another aspect, the present invention provides a method of predicting, diagnosing, monitoring, or determining Alzheimer's disease in a human, the method comprising, providing a test sample comprising a sample of bodily fluid taken from the human, determining the concentrations of three or more sample analytes in a panel of biomarkers in said sample, wherein the sample analytes are selected from the group consisting of the biomarkers in Table D, and calculating a risk score for the human using the concentrations of three or more sample analytes in the panel of biomarkers in said sample, wherein the risk score represents the probability that the human has Alzheimer's disease.

In an additional aspect, the present invention provides a method of predicting, diagnosing, monitoring, or determining Alzheimer's disease in a human. The method comprises providing a test sample comprising a sample of bodily fluid taken from the human, determining the concentrations of a panel of sample analytes in said sample, wherein the sample analytes are the biomarkers in Table A, and calculating a risk score for the human using the concentrations of sample analytes in said sample, wherein the risk score represents the probability that the human has Alzheimer's disease.

In another additional aspect, the present invention provides a method of predicting, diagnosing, monitoring, or determining Alzheimer's disease in a human. The method comprises providing a test sample comprising a sample of bodily fluid taken from the human, determining the concentrations of a panel of sample analytes in said sample, wherein the sample analytes are the biomarkers in Table B, and calculating a risk score for the human using the concentrations of sample analytes in said sample, wherein the risk score represents the probability that the human has Alzheimer's disease.

In yet another additional aspect, the present invention provides a method of predicting, diagnosing, monitoring, or determining Alzheimer's disease in a human. The method comprises providing a test sample comprising a sample of bodily fluid taken from the human, determining the concentrations of a panel of sample analytes in said sample, wherein the sample analytes are the biomarkers in Table C, and calculating a risk score for the human using the concentrations of sample analytes in said sample, wherein the risk score represents the probability that the human has Alzheimer's disease.

In still another additional aspect, the present invention provides a method of predicting, diagnosing, monitoring, or determining Alzheimer's disease in a human. The method comprises providing a test sample comprising a sample of bodily fluid taken from the human, determining the concentrations of a panel of sample analytes in said sample, wherein the sample analytes are the biomarkers in Table D, and calculating a risk score for the human using the concentrations of sample analytes in said sample, wherein the risk score represents the probability that the human has Alzheimer's disease.

In another aspect, the present invention provides a panel of biomarkers for predicting, diagnosing, monitoring, or determining Alzheimer's disease in a human, the panel comprising the biomarkers in Table A.

In yet another aspect, the present invention provides a panel of biomarkers for predicting, diagnosing, monitoring, or determining Alzheimer's disease in a human, the panel comprising the biomarkers in Table B.

In still another aspect, the present invention provides a panel of biomarkers for predicting, diagnosing, monitoring, or determining Alzheimer's disease in a human, the panel comprising the biomarkers in Table C.

In yet another aspect, the present invention provides a panel of biomarkers for predicting, diagnosing, monitoring, or determining Alzheimer's disease in a human, the panel comprising the biomarkers in Table D.

In an additional aspect, the present invention provides a method of predicting, diagnosing, monitoring, or determining Alzheimer's disease in a human. The method comprises providing a test sample comprising a sample of bodily fluid taken from the human, determining the concentrations of three or more sample analytes in a panel of biomarkers in said sample, wherein the sample analytes are selected from the group consisting of the biomarkers in Table A. Diagnostic analytes are then identified in the test sample, wherein the diagnostic analytes are the sample analytes having concentrations that are significantly different from concentrations found in a control group of humans who do not suffer from Alzheimer's disease. The concentrations of the diagnostic analytes identified are then used to calculate a risk score, wherein the risk score represents the probability that the human has Alzheimer's disease.

Other aspects and iterations of the invention are described in more detail below.

### DESCRIPTION OF FIGURES

FIG. 1 is a variable importance plot of protein biomarkers measured by the Random Forest built from the training set.

FIG. 2 depicts ROC curves for clinical variables alone and in conjunction with biomarker data.

FIG. 3 depicts a SAM plot of over and under expressed proteins in AD. The observed score (y axis) is the SAM t-statistics. Red circles indicate over-expressed proteins while green circles indicate under-expressed proteins.

FIG. 4 depicts a Venn diagram demonstrating consistency across methods for identifying altered protein expression in AD. FAS was only identified by the Wilcoxon test; FAS ligand was only identified by the SAM; prostatic acid phosphatase was identified by SAM and logistic regression but not the Wilcoxon test.

### DETAILED DESCRIPTION OF THE INVENTION

It has been discovered that a multiplexed panel of biomarkers may be used to predict, diagnose, monitor, or determine AD. The biomarkers included in the multiplexed panel are analytes known in the art that may be detected in the serum, plasma and other bodily fluids of mammals. As such, the analytes of the multiplexed panel may be readily extracted from the human in a test sample of bodily fluid. The concentrations of the analytes within the test sample may be measured using known analytical techniques such as a multiplexed antibody-based immunological assay. The combination of concentrations of the analytes in the test sample may be used to calculate a risk factor to determine whether AD is indicated in the human.

One embodiment of the present invention provides a method for predicting, diagnosing, monitoring, or determining AD in a mammal that includes determining the presence or concentration of a combination of three or more sample analytes in a test sample containing the bodily fluid of the human. The measured concentrations of the combination of sample analytes is used to calculate a risk score reflective of an AD indication in the human. Other embodiments provide computer-readable media encoded with applications containing executable modules, systems that include databases and processing devices containing executable modules configured to predict, diagnose, monitor, or determine AD in a human. Still other embodiments provide antibody-based devices for predicting, diagnosing, monitoring, or determining AD in a human.

The analytes used as biomarkers in the multiplexed assay, methods of predicting, diagnosing, monitoring, or determining AD using measurements of the analytes, systems and applications used to analyze the multiplexed assay measurements, and antibody-based devices used to measure the analytes are described in detail below.

### I. Test Samples and Biomarkers

In one aspect, the present disclosure encompasses a method for predicting, diagnosing, monitoring, or determining AD in a human. The method comprises providing a test sample comprising a sample of bodily fluid taken from the human and determining the concentrations of three or more sample analytes in a panel of biomarkers in said sample.

Components of the method are described in more detail below.

### (a) Test Sample

The method for predicting, diagnosing, monitoring, or determining AD involves determining the presence of sample analytes in a test sample. A test sample, as defined herein, is an amount of bodily fluid taken from a mammal. Non-limiting examples of bodily fluids include whole blood, plasma, serum, saliva, bile, lymph, pleural fluid, semen, perspiration, tears, mucus, CSF, and tissue lysates. In an exemplary embodiment, the bodily fluid contained in the test sample is serum. In another exemplary embodiment, the bodily fluid contained in the test sample is CSF.

A bodily fluid may be tested from any mammal known to suffer from AD or used as a disease model for AD. In one embodiment, the subject is a rodent. Examples of rodents include mice, rats, and guinea pigs. In another embodiment, the subject is a primate. Examples of primates include monkeys, apes, and humans. In an exemplary embodiment, the subject is a human. In some embodiments, the subject has no clinical signs of AD. In other embodiments, the subject has mild clinical signs of AD. In yet other embodiments, the subject may be at risk for AD. In still other embodiments, the subject has been diagnosed with AD.

As will be appreciated by a skilled artisan, the method of collecting a bodily fluid from a subject can and will vary depending upon the nature of the bodily fluid. Any of a variety of methods generally known in the art may be utilized to collect a bodily fluid from a subject. The bodily fluids of the test sample may be taken from a subject using any known device or method. Non-limiting examples of devices or methods suitable for taking bodily fluid from a mammal include urine sample cups, urethral catheters, swabs, hypodermic needles, thin needle biopsies, hollow needle biopsies, punch biopsies, metabolic cages, and aspiration. In preferred embodiments, the bodily fluid collected is blood. Methods for collecting blood or fractions thereof are well known in the art. For example, see U.S. Pat. No. 5,286,262, which is hereby incorporated by reference in its entirety. Generally speaking, irrespective of the method used to collect a bodily fluid, the method preferably maintains the integrity of the AD biomarker such that it can be accurately quantified in the bodily fluid.

### (b) The Biomarkers

One embodiment of the invention measures the concentrations of sample analytes in a panel of biomarkers within a test sample taken from a human. In this aspect, the biomarker analytes are known in the art to occur in the plasma, serum and other bodily fluids of mammals. As defined herein, the biomarker analytes include but are not limited to the biomarkers in Table A.

TABLE A

| | | |
|---|---|---|
| Adiponectin | Fibrinogen | Monocyte Chemotactic |
| | | Protein 2 |
| Adrenocorticotropic | Fibroblast Growth Factor 4 | Monocyte Chemotactic |
| Hormone | | Protein 3 |
| Agouti-Related Protein | Fibroblast Growth Factor | Monocyte Chemotactic |
| | basic | Protein 4 |
| Alpha-1-Antichymotrypsin | Follicle-Stimulating | Monokine Induced by |
| | Hormone | Gamma Interferon |
| Alpha-1-Antitrypsin | Glucagon | Myeloid Progenitor |
| | | Inhibitory Factor 1 |
| Alpha-1-Microglobulin | Glucagon-like Peptide 1, | Myeloperoxidase |
| | total | |
| Alpha-2-Macroglobulin | Glutathione S-Transferase | Myoglobin |
| | alpha | |
| Alpha-Fetoprotein | Granulocyte Colony- | Nerve Growth Factor beta |
| | Stimulating Factor | |
| Amphiregulin | Granulocyte-Macrophage | Neuronal Cell Adhesion |
| | Colony-Stimulating Factor | Molecule |
| Angiopoietin-2 | Growth Hormone | Neutrophil Gelatinase- |
| | | Associated Lipocalin |
| Angiotensin-Converting | Growth-Regulated alpha | Osteopontin |
| Enzyme | protein | |
| Angiotensinogen | Haptoglobin | Pancreatic Polypeptide |
| Apolipoprotein A-I | Heat Shock Protein 60 | Peptide YY |
| Apolipoprotein A-II | Heparin-Binding EGF-Like | Placenta Growth Factor |
| | Growth Factor | |
| Apolipoprotein A-IV | Hepatocyte Growth Factor | Plasminogen Activator |
| | | Inhibitor 1 |
| Apolipoprotein B | Immunoglobulin A | Platelet-Derived Growth |
| | | Factor BB |
| Apolipoprotein C-I | Immunoglobulin E | Pregnancy-Associated |
| | | Plasma Protein A |
| Apolipoprotein C-III | Immunoglobulin M | Progesterone |
| Apolipoprotein D | Insulin | Proinsulin |
| Apolipoprotein E | Insulin-like Growth Factor I | Intact |
| Apolipoprotein H | Insulin-like Growth Factor- | Total Proinsulin |
| | Binding Protein 2 | |
| Apolipoprotein(a) | Intercellular Adhesion | Prolactin |
| | Molecule 1 | |
| AXL Receptor Tyrosine | Interferon gamma | Free Prostate-Specific |
| Kinase | | Antigen |
| B Lymphocyte | Interferon gamma Induced | Prostatic Acid Phosphatase |
| Chemoattractant | Protein 10 | |
| Beta-2-Microglobulin | Interleukin-1 alpha | Pulmonary and Activation- |
| | | Regulated Chemokine |
| Betacellulin | Interleukin-1 beta | RANTES |
| Bone Morphogenetic | Interleukin-1 receptor | Receptor for advanced |
| Protein 6 | antagonist | glycosylation end products |
| Brain Natriuretic Peptide | Interleukin-10 | Resistin |
| Brain-Derived Neurotrophic | | |
| Factor | | |
| Calbindin | Interleukin-11 | S100 calcium-binding |
| | | protein B |
| Calcitonin | Interleukin-12 Subunit p40 | Secretin |
| Cancer Antigen 125 | Interleukin-12 Subunit p70 | Serotransferrin |
| Cancer Antigen 19-9 | Interleukin-13 | Serum Amyloid P- |
| | | Component |
| Carcinoembryonic Antigen | Interleukin-15 | Serum Glutamic |
| | | Oxaloacetic Transaminase |
| CD 40 antigen | Interleukin-1 | Sex Hormone-Binding |
| | | Globulin |
| CD40 Ligand | Interleukin-2 | Sortilin |
| CD5 | Interleukin-25 | Stem Cell Factor |
| Chemokine CC-4 | Interleukin-3 | soluble Superoxide |
| | | Dismutase 1 |
| Chromogranin-A | Interleukin-4 | T Lymphocyte-Secreted |
| | | Protein I-309 |
| Ciliary Neurotrophic Factor | Interleukin-5 | Tamm-Horsfall Urinary |
| | | Glycoprotein |
| Clusterin | Interleukin-6 | Tenascin-C |
| Complement C3 | Interleukin-6 receptor | Total Testosterone |
| Complement Factor H | Interleukin-7 | Thrombomodulin |
| Connective Tissue Growth | Interleukin-8 | Thrombopoietin |
| Factor Cortisol | | |
| C-Peptide | Kidney Injury Molecule-1 | Thrombospondin-1 |
| C-Reactive Protein | Lectin-Like Oxidized LDL | Thymus-Expressed |
| | Receptor 1 | Chemokine |
| Creatine Kinase-MB | Leptin | Thyroid-Stimulating |
| | | Hormone |
| Cystatin-C | Lipoprotein..a | Thyroxine-Binding Globulin |
| Endothelin-1 | Luteinizing Hormone | Tissue Factor |
| EN-RAGE | Lymphotactin | Tissue Inhibitor of |
| | | Metalloproteinases 1 |
| Eotaxin-1 | Macrophage Colony- | TNF-Related Apoptosis- |
| | Stimulating Factor 1 | Inducing Ligand Receptor 3 |
| Eotaxin-3 | Macrophage Inflammatory | Transforming Growth |
| | Protein-1 alpha | Factor alpha |
| Epidermal Growth Factor | Macrophage Inflammatory | Transforming Growth |
| | Protein-1 beta | Factor beta-3 |
| Epidermal Growth Factor | Macrophage Inflammatory | Transthyretin |
| Receptor | Protein-3 alpha | |
| Epiregulin | Macrophage Migration | Trefoil Factor 3 |
| | Inhibitory Factor | |
| Epithelial-Derived | Macrophage-Derived | Tumor Necrosis Factor |
| Neutrophil-Activating | Chemokine | alpha |
| Protein 78 | | |
| Erythropoietin | Malondialdehyde-Modified | Tumor Necrosis Factor beta |
| | Low-Density Lipoprotein | |
| E-Selectin | Matrix Metalloproteinase-1 | Tumor Necrosis Factor |
| | | Receptor-Like 2 |
| Factor VII | Matrix Metalloproteinase-10 | Vascular Cell Adhesion |
| | | Molecule-1 |
| FAS | Matrix Metalloproteinase-2 | Vascular Endothelial |
| | | Growth Factor |
| Fas Ligand | Matrix Metalloproteinase-3 | Vitamin D Binding Protein |
| FASLG Receptor | Matrix Metalloproteinase-7 | Vitamin K-Dependent |
| | | Protein S |
| Fatty Acid-Binding Protein | Matrix Metalloproteinase-9 | Vitronectin |
| Heart | Matrix Metalloproteinase-9, | von Willebrand Factor |
| | total | |
| Ferritin | MIF | |
| Fetuin-A | Monocyte Chemotactic | |
| | Protein 1 | |

In a preferred embodiment, the biomarker analytes are the biomarkers in Table B.

TABLE B

| | |
|---|---|
| Adiponectin | IL.1ra |
| Adrenocorticotropic.Hormone | IL.5 |
| Alpha.2.Macroglobulin | IL.7 |
| Angiopoietin.2 | IL.8 |
| Angiotensin Converting Enzyme | IL-15 |
| Apolipoprotein.CIII | Lipoprotein . . . a |
| B Lymphocyte Chemoattractant | MCP.1 |
| Beta.2.Microglobulin | MIF |
| C.Reactive.Protein | MIP.1alpha |
| CA-125 | Pancreatic.polypeptide |
| Cancer Antigen 19.9 | Prolactin |
| Carcinoembryonic Antigen | Prostatic.Acid.Phosphatase |
| Creatine.Kinase.MB | Pulmonary and Activation Regulated |
| | Chemokine |
| Eotaxin.3 | RANTES |
| Factor.VII | Resistin |
| FAS | S100b |
| Fas.Ligand | SHBG |
| Ferritin | Stem.Cell.Factor |
| Fibrinogen | Tenascin.C |
| G.CSF | Thrombopoietin |
| GRO.alpha | TIMP.1 |
| IGF.BP.2 | TNF.alpha |
| IL 10 | TNF.beta |
| IL.12p70 | VCAM.1 |
| IL.16 | Vitamin D Binding Protein |
| IL.18 | von.Willebrand.Factor |

In another preferred embodiment, the biomarker analytes are the biomarkers in Table C.

TABLE C

| | |
|---|---|
| Adrenocorticotropic.Hormone | Interleukin.8 |
| Adiponectin | MCP.1 |
| Alpha.2.Macroglobulin | MIP.1alpha |
| B.Lymphocyte.Chemoattractant . . . BLC | Pancreatic.polypeptide |
| Beta.2.Microglobulin | Prolactin |
| C.Reactive.Protein | Prostatic.Acid.Phosphatase |
| Creatine.Kinase.MB | RANTES |
| Eotaxin.3 | Resistin |
| Factor.VII | S100b |
| FAS | SHBG |
| Fas.Ligand | Stem.Cell.Factor |
| G.CSF | Tenascin.C |
| GRO.alpha | Thrombopoietin |
| IGF.BP.2 | TNF.alpha |
| Interleukin.12p70 | TNF.beta |
| Interleukin.16 | VCAM.1 |
| Interleukin.18 | Vitamin.D.Binding.Protein |
| Interleukin.1ra | von.Willebrand.Factor |

In an exemplary embodiment, the biomarker analytes are the biomarkers in Table D.

TABLE D

| | |
|---|---|
| Alpha 2 Macroglobulin | Pancreatic.Polypeptide |
| Beta 2 Microglobulin | Prolactin |
| C Reactive Protein | Prostatic.Acid.Phosphatase |
| Creatine Kinase MB | Resistin |
| Eotaxin.3 | S100b |
| FAS | Stem.Cell.Factor |
| G.CSF | Tenascin.C |
| IGF.BP.2 | Thrombopoietin |
| Interleukin.10 | TNF.alpha |
| Interleukin.15 | TNF.beta |
| linterleukin.1ra | VCAM.1 |
| Interleukin.8 | von.Willebrand.Factor |
| MIP.1alpha | |

In one aspect the present invention the number of biomarkers measured in a sample can be 3, 4, 5, 10, 25, 50, 75, 100, 125, 150, or all 194 biomarkers in Table A. In another aspect of the present invention, the number of biomarkers measured in a sample can be 3, 4, 5, 7, 9, 10, 15, 20, 25, 30, 40, 50, or all 52 biomarkers in Table B. In a further aspect of the present invention, the number of biomarkers measured in a sample can be 3, 4, 5, 6, 7, 8, 9, 10, 12, 14, 16, 18, 20, 25, 30, or all 36 biomarkers in Table C. In yet another aspect of the present invention, the number of biomarkers measured in a sample can be 3, 4, 5, 6, 7, 8, 9, 10, 12, 14, 16, 18, 20, 22, or all 25 biomarkers in Table D. In a preferred aspect of the present invention, the biomarkers measured in a sample contain at least one biomarker from Table D, more preferably, at least 3 biomarkers from Table D. The list of the number of biomarkers is not intended to be limited to the specific numbers disclosed above, as it is understood that numbers in-between the listed number of biomarkers are also included herein.

### (c) Determine Concentration (or Level) of Biomarker

The level of the biomarker may encompass the level of protein concentration or the level of enzymatic activity. In either embodiment, the level is quantified, such that a value, an average value, or a range of values is determined. In one embodiment, the level of protein concentration of three or more analytes are quantified.

There are numerous known methods and kits for measuring the amount or concentration of a specific protein in a complex sample, including ELISA, and western blot. Commercial kits include the QuantiKine ELISA kits (R&D Systems, inc.). In preferred embodiments, the method used for measuring the concentration of the biomarker is a method suitable for multiplex protein concentration determination. In an exemplary embodiment, the amount or concentration of a protein in a sample is measured using a multiplex assay device as described in Section (II) below.

In order to adjust the expected concentrations of the sample analytes in the test sample to fall within the dynamic range of the assay, the test sample may be diluted to reduce the concentration of the sample analytes prior to analysis. The degree of dilution may depend on a variety of factors including but not limited to the type of assay used to measure the analytes, the reagents utilized in the assay, and the type of bodily fluid contained in the test sample.

In one exemplary embodiment, if the test sample is human serum and the multiplexed assay is an antibody-based capture-sandwich assay, the test sample is diluted by adding a volume of diluent that is about 5 times the original test sample volume prior to analysis. In another exemplary embodiment, if the test sample is human plasma and the multiplexed assay is an antibody-based capture-sandwich assay, the test sample is diluted by adding a volume of diluent that is about 2,000 times the original test sample volume prior to analysis.

The diluent may be any fluid that does not interfere with the function of the assay used to measure the concentration of the analytes in the test sample. Non-limiting examples of suitable diluents include deionized water, distilled water, saline solution, Ringer's solution, phosphate buffered saline solution, TRIS-buffered saline solution, standard saline citrate, and HEPES-buffered saline.

### II Sample Analyte Concentration Measurement

In one embodiment, the concentration of a combination of sample analytes is measured using a multiplexed assay device capable of measuring the concentrations of up to 189 of the biomarker analytes. A multiplexed assay device, as defined herein, is an assay capable of simultaneously determining the concentration of three or more different sample analytes using a single device and/or method. Any known method of measuring the concentration of the biomarker analytes may be used for the multiplexed assay device. Non-limiting examples of measurement methods suitable for the multiplexed assay device include electrophoresis, mass spectrometry, protein microarrays, and immunoassays including but not limited to western blot, immunohistochemical staining, enzyme-linked immunosorbent assay (ELISA) methods, vibrational detection using MicroElectroMagnetic Devices (MEMS), and particle-based capture-sandwich immunoassays.

### (a) Multiplexed Immunoassay Device

In one embodiment, the concentrations of the analytes in the test sample are measured using a multiplexed immunoassay device that utilizes capture antibodies marked with indicators to determine the concentration of the sample analytes.

### (i) Capture Antibodies

In the same embodiment, the multiplexed immunoassay device includes three or more capture antibodies. Capture antibodies, as defined herein, are antibodies in which the antigenic determinant is one of the biomarker analytes. Each of the at least three capture antibodies has a unique antigenic determinant that is one of the biomarker analytes. When contacted with the test sample, the capture antibodies form antigen-antibody complexes in which the analytes serve as antigens.

In another embodiment, the capture antibodies may be attached to a substrate in order to immobilize any analytes captured by the capture antibodies. Non-limiting examples of suitable substrates include paper or cellulose strips, polystyrene or latex microspheres, a microcantiliver, and the inner surface of the well of a microtitration tray.

### (ii) Indicators

In one embodiment of the multiplexed immunoassay device, an indicator is attached to each of the three or more capture antibodies. The indicator, as defined herein, is any compound that registers a measurable change to indicate the presence of one of the sample analytes when bound to one of the capture antibodies. Non-limiting examples of indicators include visual indicators and electrochemical indicators.

Visual indicators, as defined herein, are compounds that register a change by reflecting a limited subset of the wavelengths of light illuminating the indicator, by fluorescing light after being illuminated, or by emitting light via chemiluminescence. The change registered by visual indicators may be in the visible light spectrum, in the infrared spectrum, or in the ultraviolet spectrum. Non-limiting examples of visual indicators suitable for the multiplexed immunoassay device include nanoparticulate gold, organic particles such as polyurethane or latex microspheres loaded with dye compounds, carbon black, fluorophores, phycoerythrin, radioactive isotopes, nanoparticles, quantum dots, and enzymes such as horseradish peroxidase or alkaline phosphatase that react with a chemical substrate to form a colored or chemiluminescent product.

Electrochemical indicators, as defined herein, are compounds that register a change by altering an electrical property. The changes registered by electrochemical indicators may be an alteration in conductivity, resistance, capacitance, current conducted in response to an applied voltage, or voltage required to achieve a desired current. Non-limiting examples of electrochemical indicators include redox species such as ascorbate (vitamin C), vitamin E, glutathione, polyphenols, catechols, quercetin, phytoestrogens, penicillin, carbazole, murranes, phenols, carbonyls, benzoates, and trace metal ions such as nickel, copper, cadmium, iron and mercury.

In this same embodiment, the test sample containing a combination of three or more sample analytes is contacted with the capture antibodies and allowed to form antigen-antibody complexes in which the sample analytes serve as the antigens. After removing any uncomplexed capture antibodies, the concentrations of the three or more analytes are determined by measuring the change registered by the indicators attached to the capture antibodies.

In one exemplary embodiment, the indicators are polyurethane or latex microspheres loaded with dye compounds.

### (b) Multiplexed Sandwich Immunoassay Device

In yet another embodiment, the multiplexed immunoassay device has a sandwich assay format. In this embodiment, the multiplexed sandwich immunoassay device includes three or more capture antibodies as previously described. However, in this embodiment, each of the capture antibodies is attached to a capture agent that includes an antigenic moiety. The antigenic moiety serves as the antigenic determinant of a detection antibody, also included in the multiplexed immunoassay device of this embodiment. In addition, an indicator is attached to the detection antibody.

In this same embodiment, the test sample is contacted with the capture antibodies and allowed to form antigen-antibody complexes in which the sample analytes serve as antigens. The detection antibodies are then contacted with the test sample and allowed to form antigen-antibody complexes in which the capture agent serves as the antigen for the detection antibody. After removing any uncomplexed detection antibodies the concentrations of the analytes are determined by measuring the changes registered by the indicators attached to the detection antibodies.

### (c) Multiplexing Approaches

In the various embodiments of the multiplexed immunoassay devices, the concentrations of each of the sample analytes may be determined using any approach known in the art. In one embodiment, a single indicator compound is attached to each of the three or more antibodies. In addition, each of the capture antibodies having one of the sample analytes as an antigenic determinant is physically separated into a distinct region so that the concentration of each of the sample analytes may be determined by measuring the changes registered by the indicators in each physically separate region corresponding to each of the sample analytes.

In another embodiment, each antibody having one of the sample analytes as an antigenic determinant is marked with a unique indicator. In this manner, a unique indicator is attached to each antibody having a single sample analyte as its antigenic determinant. In this embodiment, all antibodies may occupy the same physical space. The concentration of each sample analyte is determined by measuring the change registered by the unique indicator attached to the antibody having the sample analyte as an antigenic determinant.

### (d) Microsphere-Based Capture-Sandwich Immunoassay Device

In an exemplary embodiment, the multiplexed immunoassay device is a microsphere-based capture-sandwich immunoassay device. In this embodiment, the device includes a mixture of three or more capture-antibody microspheres, in which each capture-antibody microsphere corresponds to one of the biomarker analytes. Each capture-antibody microsphere includes a plurality of capture antibodies attached to the outer surface of the microsphere. In this same embodiment, the antigenic determinant of all of the capture antibodies attached to one microsphere is the same biomarker analyte.

In this embodiment of the device, the microsphere is a small polystyrene or latex sphere that is loaded with an indicator that is a dye compound. The microsphere may be between about 3 μm and about 5 μm in diameter. Each capture-antibody microsphere corresponding to one of the biomarker analytes is loaded with the same indicator. In this manner, each capture-antibody microsphere corresponding to a biomarker analyte is uniquely color-coded.

In this same exemplary embodiment, the multiplexed immunoassay device further includes three or more biotinylated detection antibodies in which the antigenic determinant of each biotinylated detection antibody is one of the biomarker analytes. The device further includes a plurality of streptaviden proteins complexed with a reporter compound. A reporter compound, as defined herein, is an indicator selected to register a change that is distinguishable from the indicators used to mark the capture-antibody microspheres.

The concentrations of the sample analytes may be determined by contacting the test sample with a mixture of capture-antigen microspheres corresponding to each sample analyte to be measured. The sample analytes are allowed to form antigen-antibody complexes in which a sample analyte serves as an antigen and a capture antibody attached to the microsphere serves as an antibody. In this manner, the sample analytes are immobilized onto the capture-antigen microspheres. The biotinylated detection antibodies are then added to the test sample and allowed to form antigen-antibody complexes in which the analyte serves as the antigen and the biotinylated detection antibody serves as the antibody. The streptaviden-reporter complex is then added to the test sample and allowed to bind to the biotin moieties of the biotinylated detection antibodies. The antigen-capture microspheres may then be rinsed and filtered.

In this embodiment, the concentration of each analyte is determined by first measuring the change registered by the indicator compound embedded in the capture-antigen microsphere in order to identify the particular analyte. For each microsphere corresponding to one of the biomarker analytes, the quantity of analyte immobilized on the microsphere is determined by measuring the change registered by the reporter compound attached to the microsphere.

For example, the indicator embedded in the microspheres associated with one sample analyte may register an emission of orange light, and the reporter may register an emission of green light. In this example, a detector device may measure the intensity of orange light and green light separately. The measured intensity of the green light would determine the concentration of the analyte captured on the microsphere, and the intensity of the orange light would determine the specific analyte captured on the microsphere.

Any sensor device may be used to detect the changes registered by the indicators embedded in the microspheres and the changes registered by the reporter compound, so long as the sensor device is sufficiently sensitive to the changes registered by both indicator and reporter compound. Non-limiting examples of suitable sensor devices include spectrophotometers, photosensors, colorimeters, cyclic coulometry devices, and flow cytometers. In an exemplary embodiment, the sensor device is a flow cytometer.

### (e) Vibrational Detection Device

In another exemplary embodiment, the multiplexed immunoassay device has a vibrational detection format using a MEMS. In this embodiment, the immunoassay device uses capture antibodies as previously described. However, in this embodiment, the capture antibodies are attached to a microscopic silicon microcantilever beam structure. The microcantilevers are micromechanical beams that are anchored at one end, such as diving spring boards that can be readily fabricated on silicon wafers and other materials. The microcantilever sensors are physical sensors that respond to surface stress changes due to chemical or biological processes. When fabricated with very small force constants, they can measure forces and stresses with extremely high sensitivity. The very small force constant of a cantilever allows detection of surface stress variation due to the binding of an analyte to the capture antibody on the microcantelever. Binding of the analyte results in a differential surface stress due to adsorption-induced forces, which manifests as a deflection which can be measured. The vibrational detection may be multiplexed. For more details, see Datar et al., MRS Bulletin (2009) 34:449-459 and Gaster et al., Nature Medicine (2009) 15:1327-1332, both of which are hereby incorporated by reference in their entireties.

### III. Predicting, Diagnosing, Monitoring, or Determining AD

In some embodiments, the method for predicting, diagnosing, monitoring, or determining AD comprises calculating a risk score for the human using the concentrations of three or more sample analytes in the panel of biomarkers in said sample, wherein the risk score represents the probability that the human has, or has the potential to develop AD. In some embodiments, a risk score greater than about 0.3 to 0.6 signifies an Alzheimer's disease diagnosis, whereas a risk score of less than about 0.3 to 0.6 signifies that the human is not diagnosed with Alzheimer's disease. In other embodiments, a risk score greater than about 0.4 to 0.5 signifies an Alzheimer's disease diagnosis, whereas a risk score of less than about 0.4 to 0.5 signifies that the human is not diagnosed with Alzheimer's disease. In a preferred embodiment, a risk score is greater than about 0.47 signifies an Alzheimer's disease diagnosis for the human, whereas when a risk score is less than 0.47 signifies that the human is not diagnosed with Alzheimer's disease. In another preferred embodiment, a risk score is greater than about 0.5 signifies an Alzheimer's disease diagnosis for the human, whereas when a risk score is less than 0.5 signifies that the human is not diagnosed with Alzheimer's disease.

The risk score may be calculated using well known statistical analysis techniques. Non-limiting examples of statistical analysis techniques that may be used to calculate the risk score include cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In preferred embodiments, the risk score may be calculated using a random forest algorithm using the concentrations of three or more sample analytes in the panel of biomarkers. In an exemplary embodiment, the risk score is calculated as described in the examples.

In some embodiments, in addition to using the concentrations of three or more sample analytes in the panel of biomarkers to calculate the risk score, the algorithm may further consider demographic variables of the human. In preferred embodiments, the variables may be selected from the group consisting of age, gender, education and APOE allele test results.

In an alternative of the embodiments, diagnostic analytes in the test sample may first be identifying, wherein the diagnostic analytes are the sample analytes having concentrations significantly different from concentrations found in a control group of humans who do not suffer from Alzheimer's disease. The risk score may then be calculated using the concentrations of the diagnostic analytes as described above.

Sample analytes having concentrations significantly different from concentrations found in a control group of humans who do not suffer from Alzheimer's disease may be identified known statistical analysis techniques. In an exemplary embodiment, a Student's t-test statistical hypothesis test is used to calculate a P-value. In some embodiments, a P-value of less than about 0.1, 0.09, 0.08, 0.07, 0.06, 0.07, 0.06, 0.05, 0.04, 0.03, 0.02 or 0.01 signifies a statistically significant difference. In a preferred embodiment, a P-value of less than about 0.049 signifies a statistically significant difference.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “multiplex analysis” refers to a type of laboratory procedure that simultaneously measures multiple analytes (dozens or more) in a single assay. It is distinguished from procedures that measure one or a few analytes at a time.

### EXAMPLES

The following examples illustrate various iterations of the invention.

### Example 1

### Identifying Biomarkers that have Diagnostic and Prognostic Utility in Alzheimer's Disease (AD)

To identify time- and cost-effective biomarkers that have diagnostic and prognostic utility in AD, biomarker data in serum collected from patients diagnosed with AD and control subjects was analyzed. Random forest analysis was utilized to create a biomarker risk score utilizing the serum-based multiplex assay results.

### Participants.

Participants included 397 individuals (197 AD, 200 controls) enrolled into the Texas Alzheimer's Research Consortium (TARC). All patients met consensus-based diagnosis for probable AD based on NINCDS-ADRDA criteria and controls performed within normal limits on psychometric assessment and were assigned a Global Score of 0 on the Clinical Dementia Rating scale. Autopsy-confirmation of clinical diagnosis was not available on study participants. The TARC project received Institutional Review Board approval at each site and all participants and/or caregivers (for AD cases) signed written informed consent documents.

Demographic characteristics of the study population are shown in Table 1. Alzheimer's patients were significantly older (p<0.001), less educated (p<0.001), and more likely to carry at least one copy of the APOE ε4 allele (p<0.001) than control participants. There were no significant differences between groups in terms of gender, race, or ethnicity, with the majority of the sample being non-Hispanic Caucasian.

TABLE 1
Participant Demographic Information

| | AD (N = 197) | Control (N = 200) | P value |
|---|---|---|---|
| Site | | | |
| Baylor | 72 (74%) | 27 | <0.0001 |
| TTUHSC | 58 (45%) | 70 | |
| UNTHSC | 33 (27%) | 91 | |
| UTSW | 34 (69%) | 15 | |
| Gender (Male) | 34.5% | 32.0% | 0.67 |
| Age (year) | | | |
| Range | 57.0-94.0 | 52.0-90.0 | <0.0001 |
| Median | 79.0 | 70.0 | |
| Education (year) | | | |
| Range | 0.0-22.0 | 10.0-25.0 | <0.0001 |
| Median | 14.0 | 16.0 | |
| APOE | | | |
| Ex/Ex | 71 | 147 | <0.0001 |
| Ex/E4 | 83 | 48 | |
| E4/E4 | 27 | 5 | |
| Unknown | 16 | 3 | |
| Hispanic Ethnicity | 3.6% | 5.4% | 0.47 |
| Race | | | |
| White | 187 | 190 | 0.67 |
| Non-white | 10 | 13 | |

Baylor = Baylor College of Medicine,
TTUHSC = Texas Tech University Health Sciences Center,
UNTHSC = University of North Texas Health Sciences Center,
UTSW—University of Texas Southwestern Medical Center

### Assays

Non-fasting blood samples were collected in serum-separating tubes during clinical evaluations, allowed to clot at room temperature for 30 minutes, centrifuged, aliquoted, and stored at −80° C. in plastic vials. Batched specimens were sent frozen to Rules Based Medicine where they were thawed for assay without additional freeze-thaw cycles. Rules Based Medicine conducted multiplexed immunoassay via the human Multi-Analyte Profile. Multiple proteins were quantified though multiplex fluorescent immunoassay utilizing colored microspheres with protein-specific antibodies. Information regarding the least detectable dose (LDD), inter-run coefficient of variation, dynamic range, overall spiked standard recovery, and cross-reactivity with other human MAP analytes can be readily obtained from Rules Based Medicine.

### Results

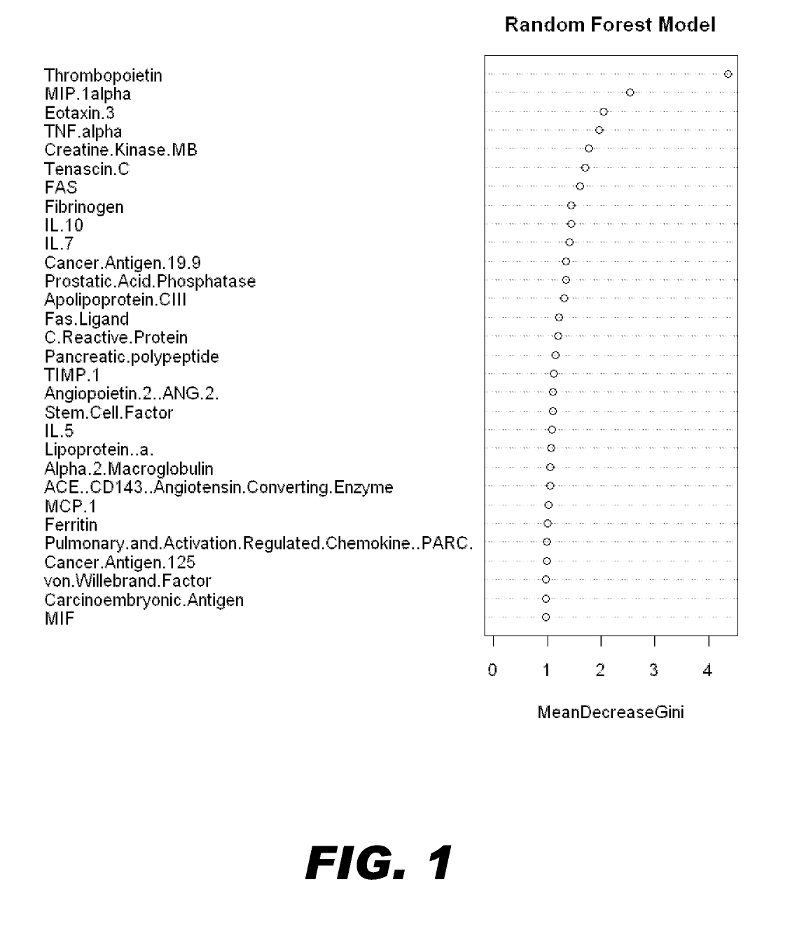

First, the subjects were randomized into either a training set or a testing set using a random number generator. Next, a random forest prediction model was built with the samples in the training set. This method has been shown to perform well in many classification and prediction scenarios, including algorithmic approaches for AD diagnostics using CSF, EEG and fMRI findings. Once the algorithm was generated with training set data, the random forest algorithm assigned a risk score to each subject in the test set that was reflective of the probability of being diagnosed with AD. That risk score was then compared with the actual diagnosis for each person in the test set, utilizing a receiver operating characteristic (ROC) curve. When the cut-off for the risk score was set at 0.47 to optimize performance (i.e. if a patient's risk score was greater than 0.47, the patient received an assignment of an AD diagnosis whereas less than 0.47 was assigned to control status), the area under the curve (AUC) for the biomarker algorithm was 0.91 (95% CI=0.88-0.95), the sensitivity and specificity was equal to 0.80 (95% CI=0.71-0.87) and 0.0.91 (95% CI=0.81-0.94), respectively. To test the robustness of the observed results against the choice of training and test sets, the randomization to training and test sets was also done by TARC site, which yielded an AUC of 0.88 demonstrating the robustness of the algorithm against choice of randomization methodology. FIG. 1 presents a variable importance plot of protein markers measured by the random-forest built from the training set.

### Example 2

### Biomarker Risk Score is a Significant, Independent Predictor of Case Status

To determine if the biomarker risk score derived in Example 1 was an independent predictor of case status (AD versus control), the following experiment was conducted. First, the biomarker data was de-correlated from the clinical variables of age, gender, education, and APOE status. Next, an additional random forest prediction model using the de-correlated biomarker data was created from the training set, which was applied to the test set for the calculation of a risk score (predicted probability of being AD). Finally, a multivariate logistic regression model was created to test the classification utility of the uncorrelated biomarker risk score as well as age, gender, education, and APOE status. As can be seen in Table 2, the biomarker risk score was a significant, independent predictor of case status.

TABLE 2
Results from logistic regression models

| | Coefficient | P-value |
|---|---|---|
| Biomarker risk score | 23.5 | 3.0E−9 |
| Age | 0.19 | 5.1E−8 |
| Gender | 0.36 | 0.013 |
| Education | −0.36 | 0.00035 |
| APOE status | 2.01 | 2.6E−6 |

### Example 3

### Biomarker Risk Score Contributes Significantly and Independently of Clinical Markers

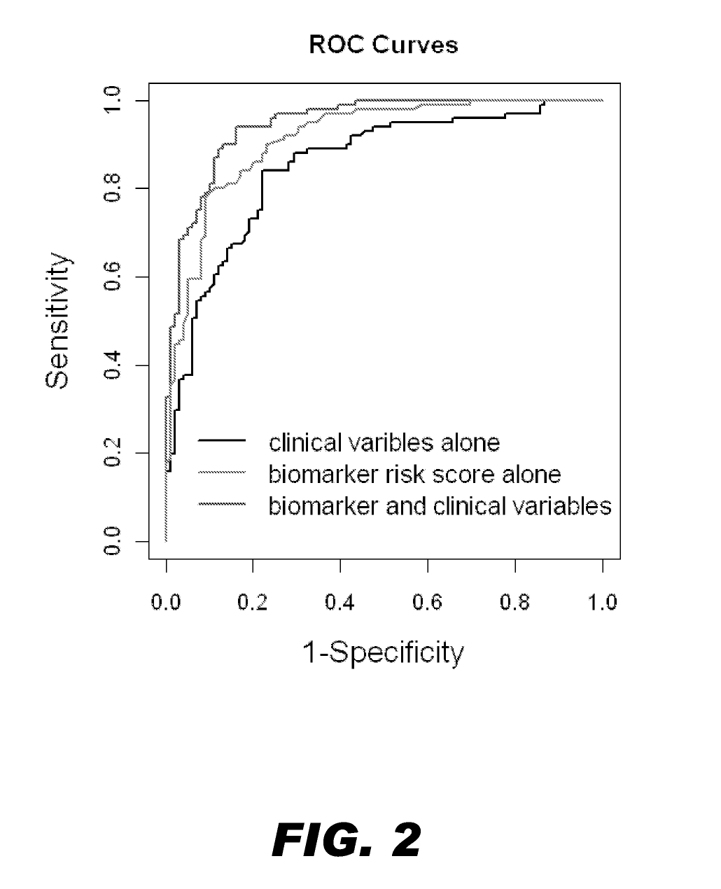

Given that age, gender, education, and APOE ε4 each are significant predictors of AD status, the next step was to determine if the biomarker risk score described above contributed significantly to and independently of the predictive utility of those clinical markers alone. To do so, logistic regression models were utilized, first with the clinical variables (age, gender, education, and APOE status) alone and then with the addition of the biomarker risk score. As would be predicted (Table 3), clinical data alone accurately classified a large portion of the sample with an observed SN=0.84 (95% CI=0.76-0.90), SP=0.78 (95% CI=0.69-0.85), and AUC=0.85 (95% CI=0.80-0.91), which was comparable to performance of the biomarker profile alone. However, addition of the biomarker data into the algorithm significantly increased the diagnostic accuracy with an observed SN=0.94 (95% CI=0.88-0.97), SP=0.84 (95% CI=0.75-0.90), and AUC=0.95 (95% CI=0.75-0.90) (FIG. 2).

TABLE 3
Diagnostic accuracy of clinical variables alone and in conjunction with
biomarker data

| | AUC | Sensitivity | Specificity |
|---|---|---|---|
| | (95% CI) | (95% CI) | (95% CI) |
| Biomarker alone | 0.91 | 0.80 | 0.91 |
| Optimal RF-based cutoff = | (0.88, 0.95) | (0.41, 0.87) | (0.81-0.94) |
| 0.47 | | | |
| Clinical variables alone | 0.85 | 0.84 | 0.78 |
| Optimal RF-based cutoff = | (0.80, 0.91) | (0.76, 0.90) | (0.69, 0.85) |
| 0.51 | | | |
| Clinical variables + biomarker | 0.95 | 0.94 | 0.84 |
| data Optimal RF-based | (0.92, 0.98) | (0.88, 0.97) | (0.75, 0.90) |
| cut-off = 0.37 | | | |

### Example 4

### Identification of Specific Proteins that were Most Predictive of Disease Status

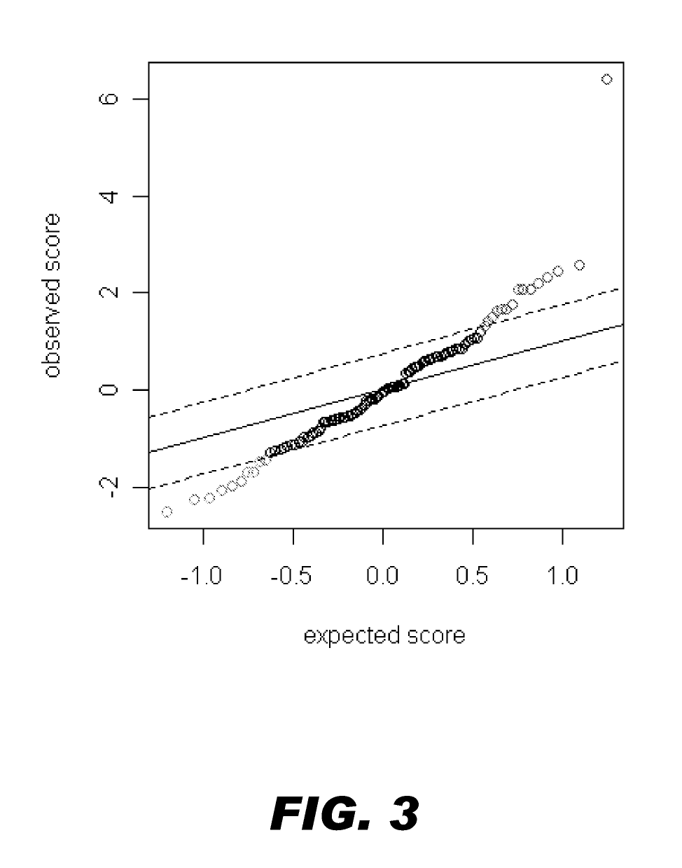

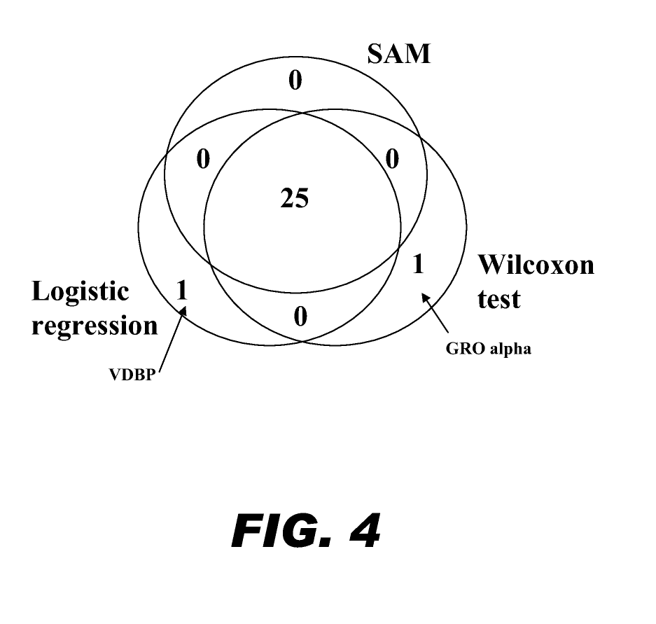

In order to identify the specific proteins that were most predictive of disease status, a SAM (significant analysis of microarray) analysis was conducted with a false discovery rate (FDR) of <0.001. There were a total of 23 proteins that were either differentially over (n=14) or under (n=9) expressed in AD (FIG. 3). Table 4 summarizes the results from the SAM analysis for each of the 23 proteins found differentially expressed in the AD group along with their fold change and risk score. In order to cross-validate the SAM procedures, Wilcoxon test and logistic regression models were utilized to identify proteins with significantly altered expression patterns. There were 22 genes identified with Wilcoxon test with a FDR less than 0.0025 and 22 from logistic regression with a FDR less than 0.01; FDR was determined using a Beta Uniform model. The FDR in Wilocoxon test and logistic regression were controlled such that they both identified similar number (22) of proteins biomarkers as that of SAM analysis. A Venn diagram demonstrating the consistency between methods utilized is shown in FIG. 4.

TABLE 4
Proteins with differential expression in AD cases
based on SAM analysis

| Protein Biomarker | SAM t-statistic | Fold Change |
|---|---|---|
| Thrombopoietin | 6.41 | 2.18 |
| Tenascin.C | 2.59 | 1.60 |
| TNF.beta | 2.46 | 1.37 |
| Eotaxin.3 | 2.33 | 1.26 |
| Pancreatic.polypeptide | 2.19 | 1.33 |
| Alpha.2.Macroglobulin | 2.09 | 2.45 |
| von.Willebrand.Factor | 2.06 | 1.29 |
| IL.15 | 2.06 | 1.26 |
| Beta.2.Microglobulin | 1.75 | 1.36 |
| VCAM.1 | 1.67 | 1.22 |
| IL.8 | 1.67 | 1.15 |
| IGF.BP.2 | 1.64 | 1.23 |
| FAS | 1.50 | 1.03 |
| Prolactin | 1.40 | 1.21 |
| Resistin | 1.33 | 1.17 |
| IL.1ra | −1.45 | 0.81 |
| Prostatic.Acid.Phosphatase | −1.49 | 0.78 |
| C.Reactive.Protein | −1.69 | 0.86 |
| TNF.alpha | −1.70 | 0.74 |
| Stem.Cell.Factor | −1.89 | 0.74 |
| MIP.1alpha | −1.97 | 0.70 |
| Creatine.Kinase.MB | −2.07 | 0.80 |
| G.CSF | −2.23 | 0.70 |
| IL.10 | −2.27 | 0.76 |
| S100b | −2.51 | 0.72 |

1-114. (canceled)

115. A method of predicting, monitoring or diagnosing Alzheimer's disease (“AD”) comprising:
a) obtaining a test sample from a human subject
b) quantifying the concentration of three or more sample biomarker analytes in a sample from the human test subject
c) comparing the concentration of the three or more sample biomarker analytes to a control concentration level of the three or more sample biomarker analytes
d) determining the human test subject has or is at risk for developing AD
wherein the at least three or more sample biomarker analytes is selected from the group comprising Alpha 2 Macroglobulin, Pancreatic Polypeptide, Beta 2 Microglobulin, Prolactin, C Reactive Protein, Prostatic.Acid.Phosphatase, Creatine Kinase MB, Resistin, Eotaxin-3, S100b, FAS, Stem Cell Factor, GCSF, Tenascin C, IGF-BP2, Thrombopoietin, Interleukin-10, TNF alpha, Interleukin-15, TNF beta, linterleukin-1ra, VCAM1, Interleukin-8, von Willebrand Factor, and MIP1 alpha.

116. The method of claim 115 wherein the test sample is whole blood, serum, plasma, or CSF.

117. The method of claim 115 wherein the test sample is serum.

118. The method of claim 115 wherein the concentrations of the sample biomarker analytes are determined using a multiplexed assay.

119. The method of claim 115 wherein the concentrations of the sample biomarker analytes are determined using ELISA.

120. The method of claim 115 wherein the determination that the human test subject has or is at risk for developing AD is based on calculating a risk score from the measure concentrations of the at least three or more sample biomarker analytes and the risk score represents the probability that the human test subject has or is at risk for developing AD.

121. The method of claim 120 wherein the risk score is calculated using a random forest algorithm.

122. The method of claim 121 wherein the algorithm further considers demographic variables of the human subject.

123. The method of claim 122 wherein the variables are selected from the group consisting of age, gender, education and APOE diagnosis.

124. A method of predicting, monitoring or diagnosing Alzheimer's disease (“AD”) comprising:
a) obtaining a test sample from a human subject
b) quantifying the concentration of three or more sample biomarker analytes in a sample from the human test subject;
c) comparing the concentration of the three or more sample biomarker analytes in the sample from the human test subject to the concentration of the three or more sample biomarker analytes in one or more samples from human control subjects that do not have AD and
d) determining the human test subject has or is at risk for developing AD
wherein the at least three or more sample biomarker analytes is selected from the group comprising Alpha 2 Macroglobulin, Pancreatic Polypeptide, Beta 2 Microglobulin, Prolactin, C Reactive Protein, Prostatic.Acid.Phosphatase, Creatine Kinase MB, Resistin, Eotaxin-3, S100b, FAS, Stem Cell Factor, GCSF, Tenascin C, IGF-BP2, Thrombopoietin, Interleukin-10, TNF alpha, Interleukin-15, TNF beta, linterleukin-1ra, VCAM1, Interleukin-8, von Willebrand Factor, and MIP1 alpha.

125. The method of claim 124, wherein the determination that the human test subject has or is at risk for developing AD is based on calculating a risk score from the measured concentrations of the at least three or more sample biomarker analytes and the risk score represents the probability that the human test subject has or is at risk for developing AD.

126. The method of claim 125, wherein the risk score for the human test subject is calculated from the concentrations of at least three or more sample biomarker analytes having concentrations that are significantly different from the concentrations found in the control subjects.

127. The method of claim 126, wherein a p-value of less than 0.049 calculated by comparing the concentrations of sample biomarker analytes from the human test subject with the one or more control subjects signifies a significant difference.

128. The method of claim 124, wherein the test sample is whole blood, serum, plasma, or CSF.

129. The method of claim 124, wherein the concentrations of the sample biomarker analytes are determined using a multiplexed assay.

130. The method of claim 124, wherein the concentrations of the sample biomarker analytes are determined using ELISA.

131. The method of claim 125, wherein the risk score is calculated using a random forest algorithm.

132. The method of claim 131, wherein the algorithm further considers demographic variables of the human subject.

133. The method of claim 115, wherein the method further comprises quantifying the concentration of one or more additional sample biomarkers which are not Alpha 2 Macroglobulin, Pancreatic Polypeptide, Beta 2 Microglobulin, Prolactin, C Reactive Protein, Prostatic.Acid.Phosphatase, Creatine Kinase MB, Resistin, Eotaxin-3, S100b, FAS, Stem Cell Factor, GCSF, Tenascin C, IGF-BP2, Thrombopoietin, Interleukin-10, TNF alpha, Interleukin-15, TNF beta, linterleukin-1ra, VCAM1, Interleukin-8, von Willebrand Factor, and MIP1alpha.

134. The method of claim 124, wherein the method further comprises quantifying the concentration of one or more additional sample biomarkers which are not Alpha 2 Macroglobulin, Pancreatic Polypeptide, Beta 2 Microglobulin, Prolactin, C Reactive Protein, Prostatic.Acid.Phosphatase, Creatine Kinase MB, Resistin, Eotaxin-3, S100b, FAS, Stem Cell Factor, GCSF, Tenascin C, IGF-BP2, Thrombopoietin, Interleukin-10, TNF alpha, Interleukin-15, TNF beta, linterleukin-1ra, VCAM1, Interleukin-8, von Willebrand Factor, and MIP1alpha.
